(19) United States
(12) Patent Application Publication
(10) Pub. No.: US 2016/0327668 A1
(43) Pub. Date: Nov. 10, 2016

(54) INTERFEROMETRY-BSED IMAGING AND INVERSION

(71) Applicant: WESTERNGECO LLC, Houston, TX (US)

(72) Inventor: Ivan Pires De Vasconcelos, Cambridge (GB)

(21) Appl. No.: 15/111,724

(22) PCT Filed: Jan. 13, 2015
(86) PCT No.: PCT/US2015/011181

§ 371 Date: Jul. 14, 2016

Related U.S. Application Data
(60) Provisional application No. 61/927,296, filed on Jan. 14, 2014.

(51) Int. Cl. G01V 1/34; A61B 8/08; G01S 15/89; A61B 8/13; G01V 1/38; G01S 13/89

(52) U.S. Cl. CPC G01V 1/34; G01V 1/3843; G01S 13/89; G01S 15/8977; A61B 8/13; A61B 8/5207; A61B 8/0866

(57) ABSTRACT

Methods and apparatuses for processing measurements to create an interferometry-based metric to measure inaccuracy of a model. The metric is used as a cost function for nonlinear inversions or simplified linear inversions or imaging.

### BACKGROUND

This disclosure relates to processing data for imaging for various industries and, in particular but not by way of limitation, relates to a new interferometry-based metric and its applications.

Imaging technologies are used in many industries to study the structures or properties of a particular object. A source may generate a wave which propagates through a media. The wave may be disturbed by the target object. A receiver may measure the disturbed wavefield by capturing some energy from the wave. From the measurements at the receiver and the source, one may obtain an image of the object or derive certain information about the properties of the object. The wave may be any kind of physical wave, such as electro-magnetic (e.g. radio wave or X-ray) or mechanical (e.g., ultrasound, acoustic or elastic). The measurements at a certain receiver may contain many limitations and other disturbances (noises) not related to the source. Sometimes, the target object is surrounded or embedded within a large, complex, but uninteresting environment. It is desirable to remove noises (or unwanted information) and to avoid limitations of any particular receivers. The measurements at receivers may be processed before they are used to produce images of the target object.

The target object under investigation may be represented directly or indirectly by a model, which may have various interesting properties. Inversion methods may be used to refine the model using the measurements made by the receiver, as described above. In such modeling, it may be desirable to establish/use a metric to indicate the accuracy of the model, even though there are limitations in the measurements.

Imaging technologies may be used in natural resource exploration, remote sensor, monitoring or surveillance, nondestructive testing, biological or medical diagnosis or treatment, etc. In all of these applications, a method or system that can remediate data acquisition limitations and improve qualities of data and resulting images or models may be used.

### SUMMARY

This summary is provided to introduce a selection of concepts that are further described in the detailed description below. This summary is not intended to identify key or essential features of the claimed subject matter, nor is it intended to be used as an aid in limiting the scope of the claimed subject matter.

In this disclosure, a metric is defined to quantify errors in a model. The metric (a test value) is based on the theories of reciprocity and interferometry for any propagating waves. This metric is defined as the norm of the difference between forward- and reverse-time fields (a forward-time test value and a reverse-time test value, respectively) that are extrapolated from observed boundary data. Because the physics behind the forward- and reverse-time extrapolation differs on how these methods use different components of the observed data, these two methods only yield the same result if the model parameters used for extrapolation are correct.

The new metric uses an acquisition geometry involving a boundary of receivers or sources that surrounds a target, with, respectively, at least one source or receiver lying outside the bounded target. The medium may be arbitrarily heterogeneous both inside and outside of the bounded target. Because of the physics of exact, reciprocity-based extrapolation, the new metric is sensitive to the model parameters inside the boundaries and is not sensitive to model parameters outside of the boundaries, i.e., the surrounding environment of the target.

The new metric may be used for non-linear waveform inversion of a target model for any type of propagating waves. The new metric may also be used for simplified or linearized migration-type imaging or inversion.

The new metric and its associated inversion methods may be used in any type of propagating waves, some examples of which include acoustic, elastic and electromagnetic wavefields, all of which are present in seismic acquisition and imaging systems, medical acquisition and imaging systems, remote sensing/surveillance systems, non-destructive imaging systems and many others.

### BRIEF DESCRIPTION OF DRAWINGS

Embodiments of this disclosure are described with reference to the following figures. The same numbers are used throughout the figures to reference like features and components. A better understanding of the methods or apparatuses can be had when the following detailed description of the several embodiments is considered in conjunction with the following drawings, in which:

FIG. 1a illustrates a data acquisition system for a marine seismic survey;

FIG. 1b illustrates a system for ultrasound imaging data acquisition, processing and display;

FIG. 1c illustrates a radio detection and ranging (RADAR) system;

FIG. 2 illustrates an imaging system with sources and receivers, and targets to be imaged wherein boundaries enclose the target;

FIGS. 3a and 3b illustrate a data field and an extrapolated field;

FIGS. 4a and 4b illustrate two systems used to test the properties of a metric in accordance with the present disclosure;

FIGS. 5a-5d illustrate extrapolated fields;

FIGS. 6a-6c illustrate the metrics, obtained in accordance with embodiments of this disclosure, for the systems shown in FIGS. 3a and 3b;

FIG. 7 illustrate a flow chart to derive a metric in accordance with this disclosure;

FIG. 8 illustrate a flow chart to perform an inversion using the metrics;

FIG. 9 illustrate a flow chart to perform a simplified inversion using the metrics; and

FIG. 10 illustrates a computer system, which may implement parts of the methods described above, in accordance with embodiments of the present invention.

### DETAILED DESCRIPTION

Reference will now be made in detail to embodiments of the present disclosure, examples of which are illustrated in the accompanying drawings and figures. In the following detailed description, numerous specific details are set forth in order to provide a thorough understanding of the subject matter herein. However, it will be apparent to one of ordinary skill in the art that the subject matter may be practiced without these specific details. In other instances, well-known methods, procedures, components and systems have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step. The first object or step, and the second object or step, are both objects or steps, respectively, but they are not to be considered the same object or step.

The terminology used in the description of the disclosure herein is for the purpose of describing particular embodiments only and is not intended to be limiting of the subject matter. As used in this description and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

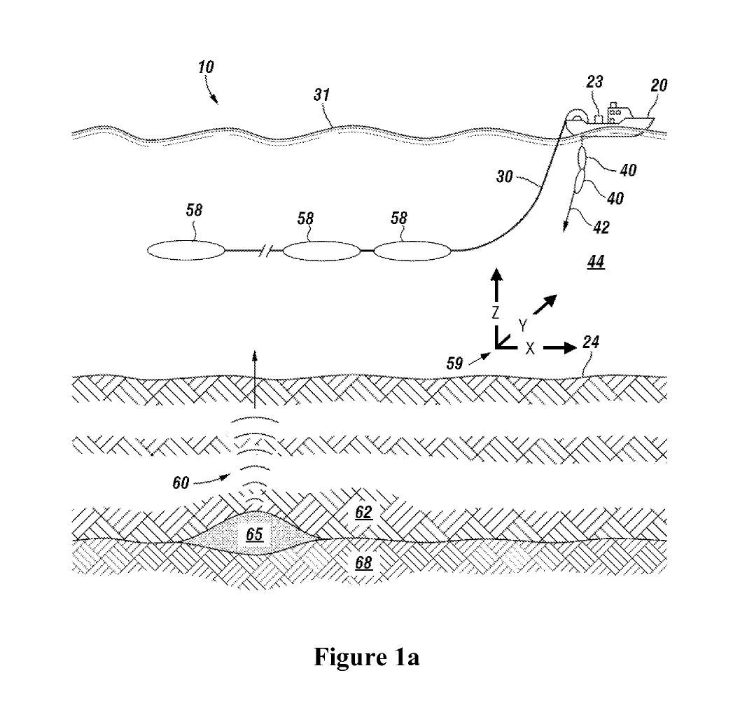

FIG. 1 illustrates three data acquisition systems in different industries. The data acquired are processed and used to construct images for various uses.

FIG. 1a illustrates a data acquisition system for a marine seismic survey. In the system 10, a survey vessel 20 tows one or more seismic streamers 30 (one streamer 30 being depicted in FIG. 1) behind the vessel 20. It is noted that the streamers 30 may be arranged in a spread in which multiple streamers 30 are towed in approximately the same plane at the same depth. As another non-limiting example, the streamers may be towed at multiple depths, such as in an over/under spread.

The seismic streamers 30 may be several thousand meters long and may contain various support cables (not shown), as well as wiring and/or circuitry (not shown) that may be used to support communication along the streamers 30. In general, each streamer 30 includes a primary cable into which seismic sensors that record seismic data are mounted. The streamers 30 contain seismic sensors 58, which may be hydrophones to acquire pressure data or multi-component sensors. For example, sensors 58 may be multi-component sensors; each sensor may be capable of detecting a pressure wavefield and at least one component of a particle motion that is associated with acoustic signals that are proximate to the sensor. Examples of particle motions include one or more components of a particle displacement, one or more components (inline (x), crossline (y) and vertical (z) components (see axes 59, for example)) of a particle velocity and one or more components of a particle acceleration.

The marine seismic data acquisition system 10 includes one or more seismic sources 40 (two seismic sources 40 being depicted in FIG. 1), such as air guns and the like. The seismic sources 40 may be coupled to, or towed by, the survey vessel 20. The seismic sources 40 may operate independently of the survey vessel 20 in that the sources 40 may be coupled to other vessels or buoys, as just a few examples.

As the seismic streamers 30 are towed behind the survey vessel 20, acoustic signals 42 (an acoustic signal 42 being depicted in FIG. 1), often referred to as “shots,” are produced by the seismic sources 40 and are directed down through a water column 44 into strata 62 and 68 beneath a water bottom surface 24. The acoustic signals 42 are reflected from the various subterranean geological formations (or targets), such as a formation 65 that is depicted in FIG. 1.

The incident acoustic signals 42 that are generated by the sources 40 produce corresponding reflected acoustic signals that are reflected by the targets, or pressure waves 60, which are sensed by the seismic sensors 58. It is noted that the pressure waves that are received and sensed by the seismic sensors 58 include “up going” pressure waves that propagate to the sensors 58 without reflection from the air-water boundary 31, as well as “down going” pressure waves that are produced by reflections of the pressure waves 60 from an air-water boundary 31.

The goal of the seismic acquisition is to build up an image of a survey area for purposes of identifying subterranean geological formations (targets), such as the geological formation 65. Subsequent analysis of the representation may reveal probable locations of hydrocarbon deposits in subterranean geological formations. Depending on the particular survey design, portions of the analysis of the representation may be performed on the seismic survey vessel 20, such as by the signal processing unit 23. In other surveys, the representation may be processed by a seismic data processing system.

FIG. 1b illustrates an ultrasound imaging data acquisition, processing and display system. The target 71 (a fetus) is to be imaged by using a transducer 72, which includes both a source and a receiver. The transmitted signal and received reflection signal (ultrasound waves 75) from the transducer 72 are sent to a processor 73. The processor 73 collects and processes the signals and converts them into a human visible image 74 and displays the image 74 on a screen. A medical care-giver may use the image 74 to monitor the condition of the fetus. In this system, the primary wave is an ultrasound wave.

FIG. 1c illustrates a radio detection and ranging (RADAR) system. The sources 81 and receivers 82 are mounted on a structure above ground (e.g. on a stationary mountain top or a moving airplane). The source signals 83 are reflected by various objects on the ground and become reflected signals 84, which are received by receivers 82. The reflected signals 84 may have various intensities due to the characteristics of the objects and time delays due to the distances. The bottom chart in FIG. 1c is an intensity (I) vs distance (X) chart in one azimuth direction. The areas 85 and 86 may indicate a mountain side and its shadow. The narrow peak 87 may indicate the metal railway tracks. The plane 88 may indicate an area of open water. By combining many reflected signals at many directions/locations, an image of the area above ground can be constructed. Using similar radio waves, a ground penetrating radar may image the subsurface structures.

For simplicity, all examples described below are related to acoustic wavefields of seismic imaging in seismic exploration in a reflective arrangement where the sources and the receivers are on the same side of the target, and where the waves emitted by sources are reflected by the target and received by receivers. The wavefields are pressure wavefields and possibly three-component velocities of particle motion. However, the methods are equally applicable to any imaging application in any arrangement, as long as the waves emitted by the sources are disturbed in some way by the target, and the disturbed waves are received by the receivers. For example, the methods are applicable in medical imaging, remote sensing, radar imaging etc. The methods are applicable for a transmission arrangement where the sources and the receivers are on the opposite sides of the target, and where the waves emitted by sources are transmitted through the target and received by receivers. The methods are applicable for mixed arrangement, where the waves emitted by sources are transmitted through the target and received by some receivers; the waves emitted by sources are also reflected by the target and received by other receivers. The different waves (propagative or dissipative), sources or receivers in different industries do not affect the wave propagation properties and the imaging processes. The wavefields can be scalar fields such as pressure fields, electric potential fields, or vector/tensor fields such as mechanical stress fields or electromagnetic fields.

In an example of seismic imaging, the subsurface target (gas or oil reservoir) can have highly complex structures, within the target or without (i.e., the overburden). The estimation of deep target properties in highly complex settings is a challenging task in exploration geophysics. On one hand, data-misfit-based full-waveform inversion methods can handle nonlinearities in the waveforms, but struggle to achieve high-resolution models from reflection data. On the other hand, image-domain tomography relies on finite-frequency information from deep reflectors, but in turn cannot easily handle nonlinear effects and requires the generation of depth-image gathers. Building on the concepts of interferometry-based imaging and exact boundary conditions, a new subsurface-domain waveform inversion metric is defined. The new metric can fully handle nonlinear effects, does not require an imaging step or image gathers, and is particularly well-suited for inversion in a target-oriented fashion. The new metric uses boundary data that enclose the target. The boundary data can be observed data, or virtual data reconstructed by various known methods.

### An Interferometric Identity for a Target Bounded Domain

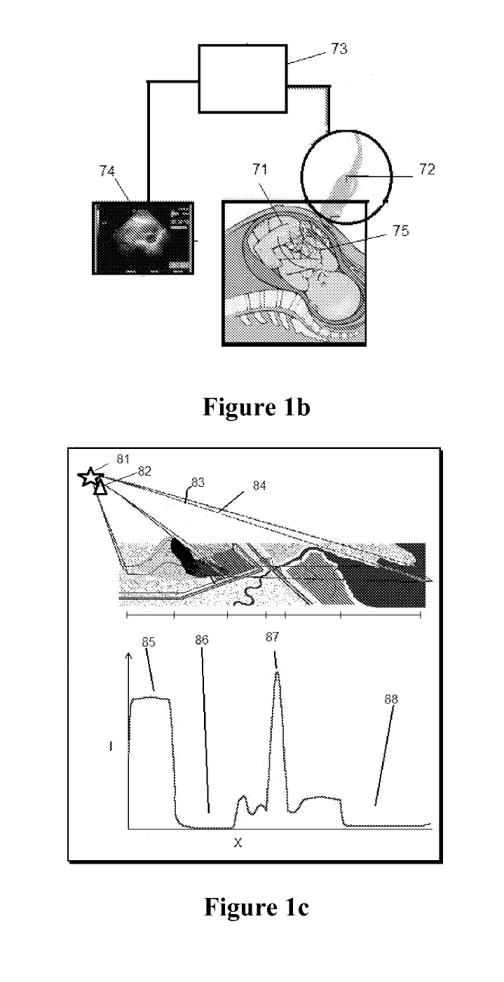

FIG. 2 illustrates a system 200. The system 200 has a top boundary ∂ [symbol] ₜ 240 and a bottom boundary ∂ [symbol] _{b} 252 that enclose a target volume 222. There are sources x_{B} 210 outside the volume 222 and x_{A} 220 inside the volume 222; and receivers x 231, 232 and 233 at the boundaries ∂ [symbol] ₜ 240 and ∂ [symbol] _{b} 252 (only receivers at boundary 240 are shown). The bottom boundary ∂ [symbol] _{b} 252 may be extended downward to become another larger bottom boundary 254.

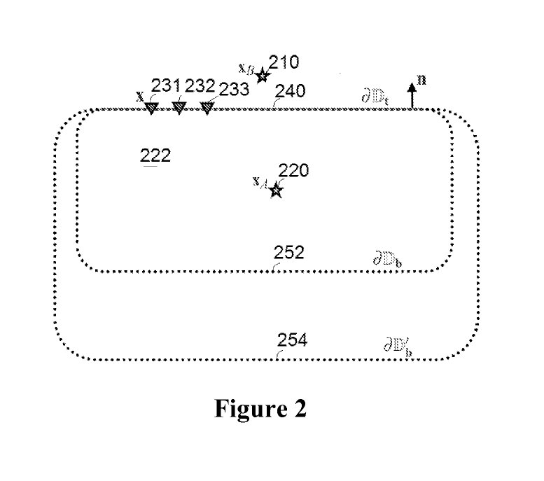

FIGS. 3a and 3b illustrate propagation paths for observed data and extrapolated data on the boundary respectively. In FIG. 3a, waves emitted by source 310 may propagate via route 336 (in-going) to be received by receivers 332 or via route 337 (out-going) to be received by the same receiver. The source 310 is outside the boundaries 340 and 350, while the receivers are on the boundaries 340 and 350, which enclose the target of interest volume 322. In FIG. 3b, the extrapolated data at the same receiver 332 is shown. The extrapolated data also has an in-going route 346 and an out-going route 347. This time the source 320 is inside the boundaries 340 and 350.

All waves in the acoustic response propagating between the points x_{B} and x_{A}, in the geometrical configuration of system 200, can be retrieved by cross-correlations via the integral:

$$\hat{p} ( {x}_{A} , {x}_{B} ) = {\int }_{\partial 𝔻} \frac{1}{i ω ρ} [ {\partial }_{n} \hat{p} ( x , {x}_{B} ) {\hat{G}}^{*} ( x , {x}_{A} ) - \hat{p} ( x , {x}_{B} ) {\partial }_{n} {\hat{G}}^{*} ( x , {x}_{A} ) ] {d}^{2} x , \qquad ( 1 )$$

where the fields in the integrand are the superposition of all the in- and out-going waves shown in FIGS. 3a and 3b.

Throughout this disclosure, with wavefield extrapolation/redatuming in mind, it may be assumed that all \hat{p} fields correspond to “boundary data,” which are either observed directly or indirectly reconstructed. \hat{p} fields may be subsequently used for extrapolation and imaging/inversion. All Ĝ fields are full-wave “wavefield extrapolators,” and denote either explicitly or implicitly the impulse response of a wavefield modelling operator of choice (e.g., (e.g., ray-tracing, finite differences, spectral elements), given a subsurface model. The notation ∂ₙ indicates local normal derivatives. The * superscript denotes a complex conjugation in the frequency domain, thus corresponding to a cross-correlation in the time domain. Here, the geometrical configuration in FIG. 3 also allows for the retrieval of \hat{p}(x_{A},x_{B}) by means of cross-convolution:

$$- \hat{p} ( {x}_{A} , {x}_{B} ) = {\int }_{\partial 𝔻} \frac{1}{i ω ρ} [ {\partial }_{n} \hat{p} ( x , {x}_{B} ) \hat{G} ( x , {x}_{A} ) - \hat{p} ( x , {x}_{B} ) {\partial }_{n} \hat{G} ( x , {x}_{A} ) ] {d}^{2} x . \qquad ( 2 )$$

It is recognized that the correlation- and convolution-based integrals in Eqs. 1 and 2 retrieve the exact same response (with opposite polarity). Thus, by adding Eq. 1 and 2, the following identity is obtained:

$$I = {\int }_{\partial 𝔻} \frac{1}{i ω ρ} [ {\partial }_{n} {\hat{p}}_{B} 2 ℜ \{ {\hat{G}}_{A} \} - {\hat{p}}_{B} 2 ℜ \{ {\partial }_{n} {\hat{G}}_{A} \} ] {d}^{2} x = 0 , \qquad ( 3 )$$

where \hat{p}_{B} and Ĝ_{A} are now shorthand for the fields in Eq. 1. Physically, this identity simply states that both forward- (convolution-based) and reverse-time (correlation-based) extrapolation yield the same fields. This identity I and its variants may be referred to as “interferometric identities.”

In the above equations 1-3, the boundaries are assumed to be the same. This assumption is made merely for the purpose of simplicity, and not by necessity. In fact, as illustrated in FIG. 2, the boundaries for forward-extrapolation (240 and 252) and those for the reverse-extrapolation (240 and 254) do not have to be the same and/or coincide. The only requirement is that outside sources x_{B} 210 are outside the boundaries and inside sources x_{A} 220 are inside the boundaries. There is no limitation on the shape of the boundaries.

It is noted that although forward- and reverse-time extrapolation yield the same final result, they each rely on different physical contributions from the boundary data. To illustrate this, referring to FIG. 3a, given data with both in- 336 and out-going 337 wave components, if we choose an extrapolation model that is homogeneous outside the bounded domain 340 and 350, Eq. 3 can be recast as

$$I = {I}_{hom} = {\int }_{\partial 𝔻} \frac{2}{i ω ρ} [ ( {\hat{p}}_{B}^{( out )} ) {( {\partial }_{n} {\hat{G}}_{A}^{( out )} )}^{*} + ( {\hat{p}}_{B}^{( i n )} ) ( {\partial }_{n} {\hat{G}}_{A}^{( out )} ) ] {d}^{2} x , \qquad ( 4 )$$

where, from the contributions in the integrand, we see that forward-time (convolution) extrapolation uses only the in-going waves \hat{p}_{B}⁽ⁱⁿ⁾ from the data, whereas reverse-time (correlation) extrapolation uses only the out-going waves \hat{p}_{B}^{(out)}.

One advantage of the identities discussed above is that they can be quite flexible in terms of which geometries and/or boundary conditions can be employed. One feature of Eq. 1 is that the full integration surface is composed of two parts: a top boundary ∂ [symbol] ₜ 240 and a bottom boundary ∂ [symbol] _{b} 252. For a moment, we assume that all wave responses related to sources on top boundary ∂ [symbol] ₜ 240 are observable, while those related to sources on bottom boundary ∂ [symbol] _{b} 252 are not and thus constitute “missing boundary data.” In this case, it is possible to arrive at an integral identity that relates the missing data boundary bottom boundary 252 with observed data related to the top boundary 252. To that end, we again sum Eqs. 1 and 2 to retrieve our integral identity in Eq. 3. Next, we let the convolution boundary 252 go to infinity, so that it vanishes under far-field radiation conditions. Then, after rearranging terms according to their integration surfaces, the following integrals are obtained:

$$\begin{matrix}{I}_{top} = {\int }_{\partial {𝔻}_{t}} \frac{1}{i ω ρ} [ {\partial }_{n} {\hat{p}}_{B} 2 ℜ \{ {\hat{G}}_{A} \} - {\hat{p}}_{B} 2 ℜ \{ {\partial }_{n} {\hat{G}}_{A} \} ] {d}^{2} x & ( 4 b ) \\ {I}_{bot} = {\int }_{\partial {𝔻}_{b}} \frac{1}{i ω ρ} [ {\partial }_{n} {\hat{p}}_{B} {\hat{G}}_{A}^{*} - {\hat{p}}_{B} {\partial }_{n} {\hat{G}}_{A}^{*} ] {d}^{2} x , and & \\ {I}_{top} + {I}_{bot} = 0 & \end{matrix}$$

Eq. 4b is simply a restatement of Eq. 3 in terms of the separate integral contributions of the boundaries 240 and 252, under the implicit assumption that there are no in-going waves arriving at the boundary 254 once that is set to infinity. However, because in this case the boundary 252 can be thought of as a “missing data boundary” (i.e., a boundary where data are desired but are not physically available), this form (i.e., Eq. 4b) of the interferometric integral identity is of use for certain practical acquisition configurations, e.g., seismic imaging.

### Subsurface-Domain Objective Function

The interferometric identity in Eq. 3 only holds for the correct extrapolator Ĝ_{A} that satisfies both integral relations in Eqs. 1 and 2. Therefore, for a given set of observed \hat{p}_{B} fields, using any trial model m that yields Ĝ_{A}″≠Ĝ_{A} would result in:

$$I = {\int }_{\partial 𝔻} \frac{1}{i ω ρ} [ {\partial }_{n} {\hat{p}}_{B} 2 ℜ \{ {\hat{G}}_{A}^{′} \} - {\hat{p}}_{B} 2 ℜ \{ {\partial }_{n} {\hat{G}}_{A}^{′} \} ] {d}^{2} x \neq 0. \qquad ( 5 )$$

As such, we can immediately define the metric

$$J ( {x}_{A} , {x}_{B} ; m ) = {‖ {\int }_{\partial 𝔻} \frac{1}{i ω ρ} [ {\partial }_{n} {\hat{p}}_{B} 2 ℜ ( {\hat{G}}_{A}^{′} ( m ) \} - {\hat{p}}_{B} 2 ℜ \{ {\partial }_{n} {\hat{G}}_{A}^{′} ( m ) \} ] {d}^{2} x ‖}^{2} , \qquad ( 6 )$$

which, according to the exact interferometric relation in Eq. 3, will be a minimum (i.e., equal to zero) when m is the model that produces the correct Ĝ_{A}. Merely by way of example, one of the numerous advantages of this new metric is that different configurations may be chosen for medium parameters to perform extrapolation outside the bounded volume. For boundary data \hat{p}_{B} in arbitrary media, the medium outside the volume may be chosen to be to be homogeneous, so that the metric implicitly yields:

$$J ( {x}_{A} , {x}_{B} ; m ) = {‖ {\int }_{\partial 𝔻} \frac{2}{i ω ρ} [ ( {\hat{p}}_{B}^{( out )} ) {( {\partial }_{n} {\hat{G}}_{A}^{′ ( out )} ( m ) )}^{*} + ( {\hat{p}}_{B}^{( i n )} ) ( {\partial }_{n} {\hat{G}}_{A}^{′ ( out )} ( m ) ) ] {d}^{2} x ‖}^{2} . \qquad ( 7 )$$

In this case, because the metric uses only the outgoing extrapolator Ĝ_{A}″^{(out)}, it relies on all components of the data (acquired in a fully heterogeneous medium) but is sensitive only to model parameters inside the bounded domain.

The metric in Eq. 7 is minimum or zero when the model m is correct, so Eq. 7 can be directly used for the optimization objective:

$$minimize {\sum }_{all {x}_{B}} {\sum }_{all {x}_{A}} J ( {x}_{A} , {x}_{B} ; m ) , for m ( {x}_{m} ) , {x}_{m} ∈ 𝔻 \qquad ( 8 )$$

where volume [symbol] is the target volume bounded by boundary ∂ [symbol] and xₘ are the locations within the target volume where the model parameters are to be updated. Thus Eq. 8 allows for the design of a full-waveform imaging/inversion scheme. Such schemes are in principle fully nonlinear in m(xₘ), because both boundary data as well as extrapolator fields are themselves nonlinear with respect the subsurface model. Note that the quantity in Eq. (8) results in a single value for a given frequency; thus it is frequency- or time-dependent. When multiple frequency and/or time samples are available, Eq. (8) can be modified to include a summation over all time or frequency samples. When used for minimization, the objective function in Eq. (8) can be used with additional terms such as regularization and pre-conditioning terms, as in common practice in any inversion procedure based on optimizing a cost function.

The identities or metrics in Eqs. 6, 7 and 8 assume that the data on the enclosing boundary ∂ [symbol] are acquired or known. There are situations where the boundary data at some portions of the enclosing boundary ∂ [symbol] are not known. For example, for the system in FIG. 2 where the far-field radiation conditions areapplied to the convolution boundary ∂ [symbol] ′_{b}, J can be recast in terms of Eq. 4b. The identity in Eq. 7 becomes:

$$\begin{matrix}J ( {x}_{A} , {x}_{B} ; m ) = {J}^{′} ( {x}_{A} , {x}_{B} ; m ) = \\ = ‖ {\int }_{\partial {𝔻}_{t}} \frac{1}{i ω ρ} [ {\partial }_{n} {\hat{p}}_{B} 2 ℜ \{ {\hat{δ G}}_{A} ( m ) \} - {\hat{p}}_{B} 2 ℜ \{ {\partial }_{n} δ {\hat{G}}_{A} ( m ) \} ] {d}^{2} x + \\ { {\int }_{\partial {𝔻}_{b}} \frac{1}{i ω ρ} [ {\partial }_{n} {\hat{p}}_{B} δ {\hat{G}}_{A}^{*} ( m ) - {\hat{p}}_{B} {\partial }_{n} δ {\hat{G}}_{A}^{*} ( m ) ] {d}^{2} x ‖}_{2}^{2} ,\end{matrix} \qquad ( 9 )$$

where each term here corresponds to a different integral quantity over a different portion of the enclosing boundary. This particular form of J is suitable for defining a metric in the case when the observed data is only available at a portion of the boundary. If it is assume that the data fields now \hat{p}_{B} and ∂ₙ\hat{p}_{B} are observed only over the top boundary 240, one possible modification to the metric in Eq. 9 is:

$$( 10 )$$

$$\begin{matrix}J ( {x}_{A} , {x}_{B} ; m ) = {J}_{min}^{′} ( {x}_{A} , {x}_{B} ; m ) = \\ = ‖ {\int }_{\partial {𝔻}_{t}} \frac{1}{i ω ρ} [ {\partial }_{n} {\hat{p}}_{B} 2 ℜ \{ δ {\hat{G}}_{A} ( m ) \} - {\hat{p}}_{B} 2 ℜ \{ {\partial }_{n} δ {\hat{G}}_{A} ( m ) \} ] {d}^{2} x + \\ { {\int }_{\partial {𝔻}_{b}} \frac{1}{i ω ρ} [ {\partial }_{n} {\hat{p}}_{B}^{mod} ( m ) δ {\hat{G}}_{A}^{*} ( m ) - {\hat{p}}_{B}^{mod} ( m ) {\partial }_{n} δ {\hat{G}}_{A}^{*} ( m ) ] {d}^{2} x ‖}_{2}^{2}\end{matrix}$$

where \hat{p}_{B}^{mod} and now ∂ₙ \hat{p}_{B}^{mod} are “predicted data,” modeled using the trial model m. It is straightforward to verify that if m is the correct model, then the Js in Eq. 10 are zero, because in that case \hat{p}_{B}^{mod}=\hat{p}_{B}, ∂ₙ\hat{p}_{B}^{mod}=∂ₙ\hat{p}_{B} and ∂ₙδĜ_{A}(m)=0, so Eq. 3 is satisfied. Other than the fact that part of the data used in Eq. 10 are “predicted data” (or estimated using the model m), the identity J or J′ of Eq. 10 is equivalent to the identity defined in Eq. 6 or 7. It can be used as a metric to measure the accuracy of the model m. It may also be used to define a cost function or optimization objective as in Eq. 8. It is noted, however, that from an inverse problem point of view the metrics in Eqs. 9 and 10 are substantially different: while Eq. 9 relies on known data over the enclosing boundary to solve for medium parameters, Eq. 10 uses limited data to jointly update the model and predict (estimating) the missing boundary data.

### Numerical Example

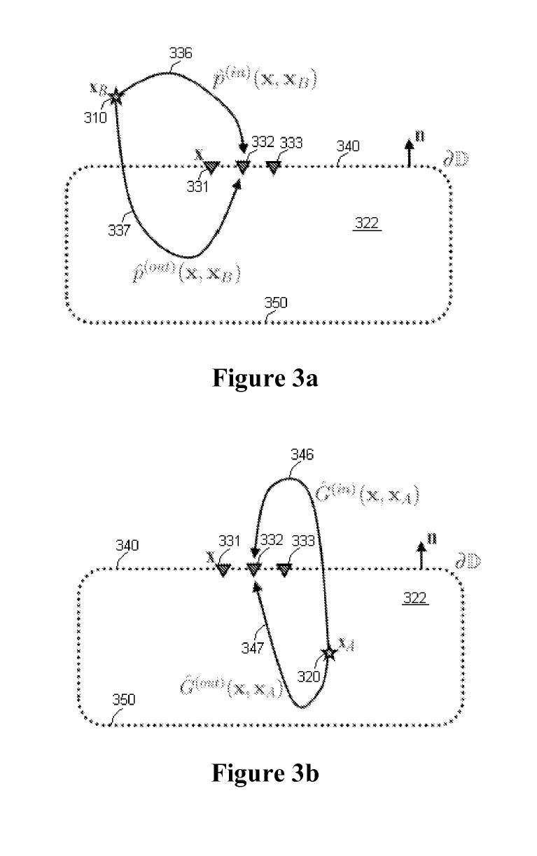

FIGS. 4a and 4b illustrate two systems 400 and 490 that are used to show properties and utilities of the metric discussed above. Most parts of the two systems 400 and 490 are the same: boundaries 440 and 450 enclose target volume 414 and 454 respectively; source 410 is outside the boundaries; source 420 is inside the boundaries. The target 414 and 454 are the same; both targets are inhomogeneous. However, the media 415 and 416, which are outside the boundaries, are different: medium 415 is inhomogeneous while medium 416 is homogeneous. Boundary data are acquired on the top boundaries 440 using receivers 431.

Here, the boundary data used in all extrapolation examples are acquired using the model in FIG. 4a; i.e., the data contain all scattering interactions with the inhomogeneities inside and outside the bounded domain. Using a single, fixed source position at x_{B} in all of the examples, with receivers at discrete positions x, uniformly sampled at 7.6 meters along the boundary 440 (FIG. 4a or 4b), recording pressure and both components of particle velocity. The source excitation function is a zero phase Ricker wavelet with 15 Hz peak frequency. Here, wavefield extrapolation is done by “wavefield injection,” following the vector-acoustic extrapolation scheme.

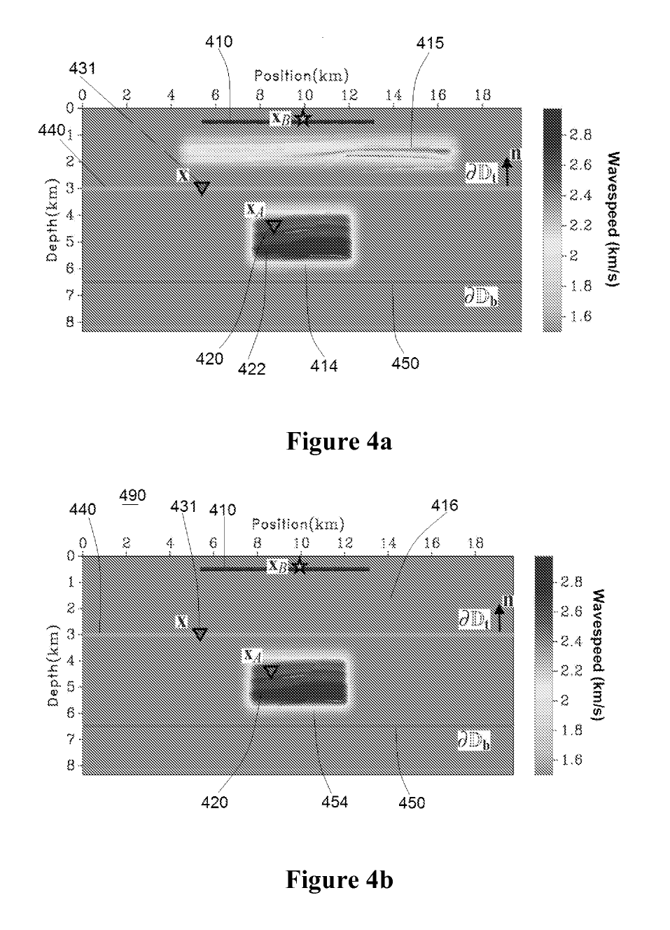

FIGS. 5a-5d illustrate the wavefields at receivers and the corresponding interferometry-based quantities. FIG. 5a shows the extrapolated fields at a receiver line at 4.64 km depth in the system 400 shown in FIG. 4a; FIG. 5c shows the extrapolated fields at a receiver line at 4.64 km depth in the system 490 shown in FIG. 4b; FIGS. 5b and 5d show the corresponding identities. Keeping in mind that the input boundary data for all panels in FIG. 5 are the same (i.e., acquired with the model in FIG. 4a), therein it can be shown that the forward-time (i.e., convolution-type; Eq. 2) extrapolated fields and the corresponding evaluated interferometric identity (Eq. 3) using different extrapolation models outside the bounded domain. When comparing the extrapolated fields in FIGS. 5a and 5c, it is virtually impossible to distinguish which field was extrapolated using the model in FIG. 4a and which uses the homogeneous-exterior model of FIG. 4b. And likewise, although there are some minor observable differences between FIGS. 5b and 5d, the same can be said of the result of the interferometric identity, using either of the two different models in FIG. 4a or 4b. Here, it is important to note that the vector-acoustic implementation of both reverse- and forward-time extrapolation (Eqs. 1 and 2) naturally accounts for the correct handling of in- and out-going waves at the boundary, and thus the decomposition in Eq. 7 is implicitly carried out in the numerical evaluation of Eq. 6.

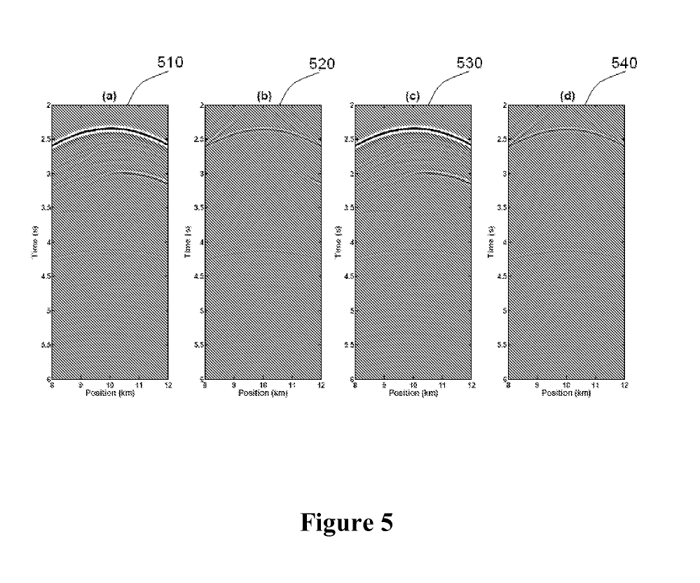

The equivalence of the models of FIG. 4a or 4b for extrapolation is further supported by the evaluation of the interferometric misfit J(xA,xB) in Eq. 6 for x_{A} varying over a target volume (FIG. 6). FIGS. 6a-6c illustrate the interferometric misfit over a target subvolume within the bounded domain. The observed data are the same for all three panels: acquired in the medium shown in FIG. 4a. FIG. 6a shows the result of the interferometric misfit using the model 400 in FIG. 4a, while FIG. 6b shows the same using the model 490 in FIG. 4b. FIG. 6c shows the result using a model, obtained by spatial smoothing of the object 454 in the model 490 in FIG. 4b, that is incorrect inside the bounded domain. The main difference between model 400 and model 490 is that the objects 415 and 416 are outside the boundaries. It is apparent that the results 610 and 620 are almost the same, which means the interferometric is insensitive to the changes in objects (i.e. 415 versus 416) outside the boundaries. The differences between results 620 and 630 are significant, even though the model differences are quite minor. In FIG. 6c, light colors indicate misfit increases only due to model changes in the interior of the bounded domain, with no actual need for the exterior model parameters present in the actual configuration where the data are acquired (FIG. 4a). The difference in the models for results 620 and 630 is only the difference between the object 454 and the “smoothed” object 454. Therefore, the interferometric identity is very sensitive to the misfit of the model of an object inside the boundaries.

This observation verifies an aspect of interferometric misfit in Eq. 6. If the model properties inside the target domain are correct, the J-metric is minimum. The model properties outside the target domain are irrelevant. Because of this feature, the interferometric misfit can be used to detect model errors inside the boundary for data collected using the model in FIG. 4a, without knowledge of parameters outside the boundary.

As discussed above, the novel metric can quantify errors in subsurface models using wavefield extrapolation schemes based on convolution- and correlation-type reciprocity. In aspects of the present disclosure, a subsurface-domain metric for inversion may be based on the difference between forward- and reverse-time extrapolated fields. The new metric may rely on full-waveform data and extrapolators, and therefore may allow for nonlinear inversion of full-waveform data.

The proposed subsurface-domain misfit, in accordance with an embodiment of the present disclosure, has features that distinguish it from existing tomography/inversion metrics. Although it is defined in the subsurface domain, the interferometric misfit is not a depth image-based metric, as it does not require the evaluation of an imaging condition or of depth-domain image gathers of any kind. As shown here, the misfit may be evaluated in the subsurface domain even for a single source point, though it can also take advantage of multiple source locations. In addition, being well-defined in both the frequency- and time-domain, the metric may allow for multi-scale inversion approaches in the subsurface domain.

Another feature of the metric, in accordance with an embodiment of the present invention, is that it may be used in a target-oriented fashion: given sufficient enclosing boundary data acquired in an arbitrarily heterogeneous medium, the proposed interferometric misfit may be used to quantify subsurface errors on the inside of a target bounded domain, with no knowledge of the medium properties outside it. This feature, which holds for arbitrarily complex models, may be used for applications such as targeted reservoir characterization and localized time-lapse monitoring using waveform information.

### Method to Derive the Metric

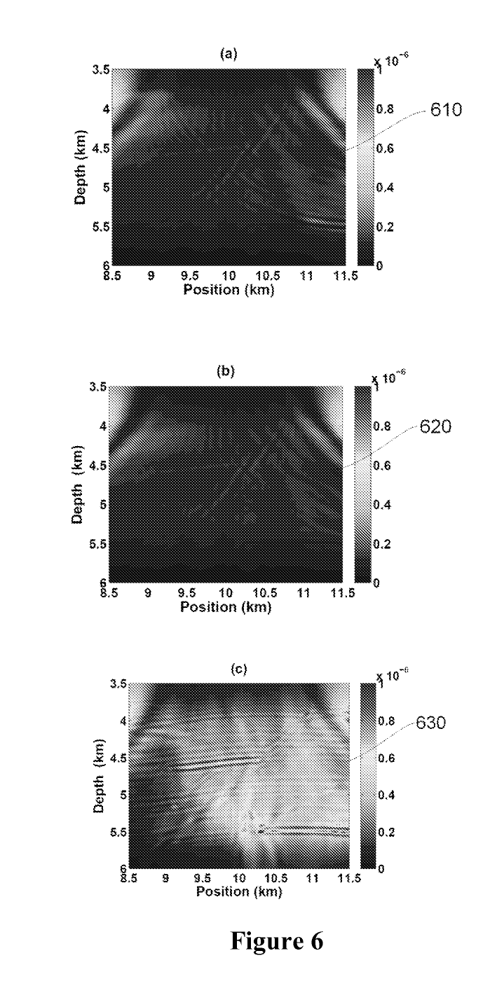

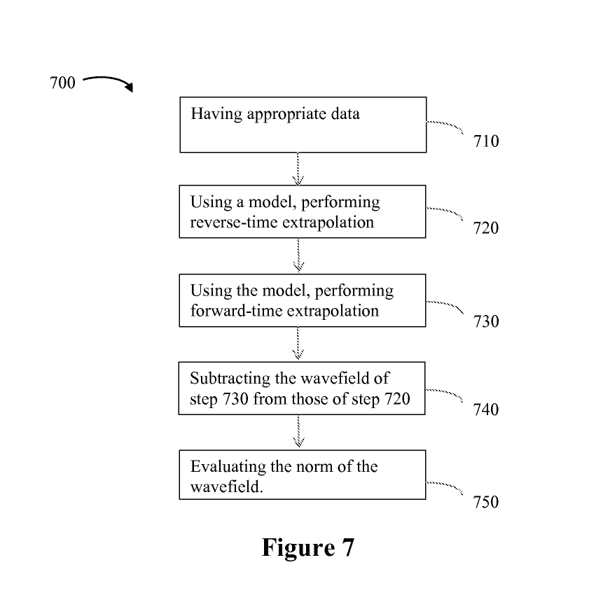

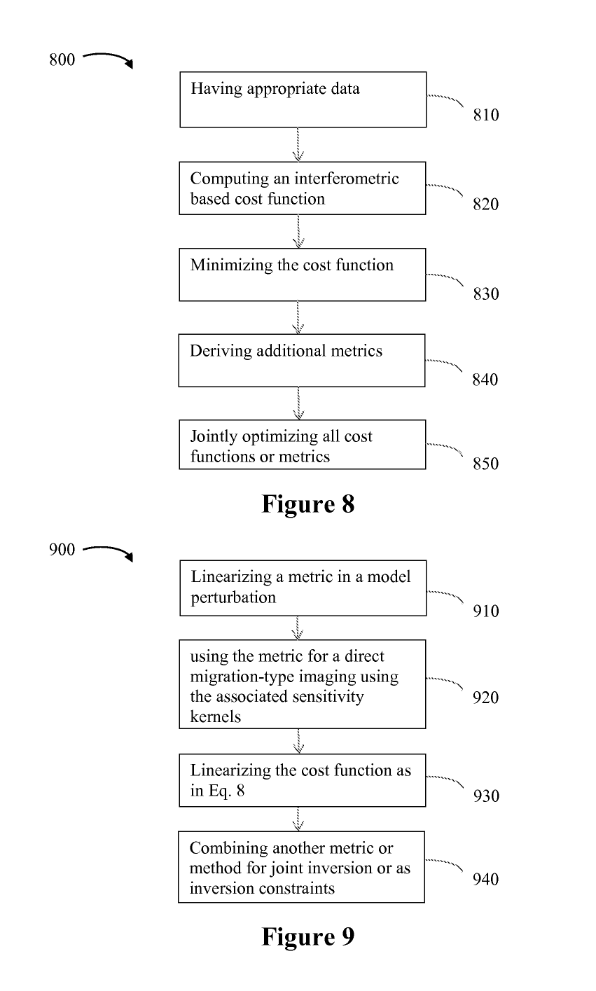

FIG. 7 provides a method 700 for deriving the metric as defined in Equations 6 or 7, in accordance with an embodiment of the present disclosure.

At step 710 data is received on an enclosing boundary of receivers associated with at least one source outside the boundary.

At step 720, using a model of the volume, performing reverse-time (or correlation-type) extrapolation of the boundary data to generate wavefields for locations in the volume within the boundary (the wavefield magnitude at a location from this step may be referred to as a reverse-time test value).

At step 730, using the model of the volume, performing forward-time (or convolution-type) extrapolation of the boundary data to generate wavefields for locations in the volume within the boundary (the wavefield magnitude at a location from this step may be referred to as a forward-time test value).

At step 740, subtracting the wavefields of step 730 (the forward-time test value) from those of step 720 (the reverse-time test value) for locations in the volume within the boundary. These differences (which may be referred to as test values) are the metrics which indicate the correctness of the model that represents the volume inside the boundaries.

At step 750, evaluating the norm of the quantities of step 740 at subsurface locations of interest, which indicate the correctness of the model at that location and other locations.

The steps mentioned above are listed for illustration purposes. It is understood that not all steps are necessary (for example, steps 750 may be omitted), and steps do not have to be performed in a particular sequence (for example, step 730 may be performed before step 720 or simultaneously).

Here, “appropriate data” as mentioned in step 710 can be any data that is related to the enclosing boundaries. They can be observed data from receivers on the enclosing boundaries. They may be virtual data from virtual receivers on the enclosing boundaries which are indirectly derived from observed data using a variety of methods, such as data driven iterative Marchenko-based autofocussing methods, or inverse-scattering-series methods. They may comprise a mixture of the above two data, i.e., partially from observed data from receivers on the portion of the enclosing boundaries, and partially from virtual data from virtual receivers on the remaining portion of the enclosing boundaries. A portion of the “appropriate data” may even be derived by means of model-dependent simulation.

In the seismic imaging example, e.g., in an acoustic system, the data may consist of all necessary field components, e.g., pressure fields plus 3-component acceleration. In an elastic system, the data may consist of all necessary field components separated into components of in- and out-going waves on the boundary, e.g., all in- and out-going waves from all P- and S-wave components. As discussed above, the target object can be homogeneous or arbitrarily heterogeneous, isotropic or anisotropic, or lossless or possibly attenuative. For examples other than seismic imaging, the data are those appropriate wavefield measurement data.

Depending on the type of the wavefield, e.g. acoustic, elastic or electromagnetic, the metrics (the test values) may be a scalar, a vector, a tensor or other appropriated values. The metric may be a single value for a pressure wavefield at a location. The metric may have multiple values, e.g. as in a vector for a velocity wavefield.

There are many ways to perform forward- (convolution type) or reverse (correlation type) extrapolation of boundary data. For demonstrative purposes with no limitations, the following are some common methods for reverse-time/forward-time extrapolation of the boundary data where the methods:
a. can be performed with any type of extrapolators, e.g., full-wave-equation (e.g., finite differences, spectral elements, finite elements), beam-based (e.g., Gaussian beams), ray-based (e.g., ray or wavefront tracing);
b. can be performed implicitly (e.g., by wavefield injection) or with the explicitly computed impulse response of an extrapolator)
c. can be equally performed in the time- or frequency-domain, in the context of items a) and b);
d. can be performed, within items a) to c), using the exact reciprocity integral formulations according to the physical system in question (e.g., acoustic, elastic, electromagnetic): e.g., in the acoustic case this corresponds to exact reverse-time Vector-Acoustic extrapolation (U.S. patent application Ser. No. 14/112,891, Attorney docket No. IS11.0173 and U.S. patent application Ser. No. 13/345,412, Attorney docket No. IS10.0934);
e. can be designed to include heterogeneities outside the boundary; or
f. can be designed with a model that is homogeneous outside of the boundary.

The interferometric misfit we present here, J in Eq. 7, is shown to be a reliable metric for quantitative evaluation of errors in subsurface models. This is a new metric that can be used for waveform-based inversion. While J is a type of waveform misfit, it is important to note that it does not fall under the class of “data-domain” misfit functions used in the more mainstream full-waveform inversion (FWI) approaches. This is, firstly, because this metric operates on quantities in the subsurface domain as opposed to data-domain metrics of FWI. Secondly, the quantities in question are nonlinearly related to the observed boundary data (i.e., the extrapolators are nonlinear in the model), and the computation of our interferometric identity departs substantially from the forward modeling step of FWI.

Although our interferometric misfit is a subsurface-domain metric, we note that it is not an “image-domain” metric; i.e., it does not fall into the general category of subsurface metrics that are based on a depth image as output of some form of migration/inversion scheme. In fact, the majority of subsurface-domain objective functions available in the literature operate on depth-imaged seismic data, most often in the context of an “extended image” or “extended model.” Some examples of “extended image” or “extended model” include: wave-equation migration velocity analysis, differential-semblance optimization in the extended model space, angle-domain finite-frequency tomography and extended-image inversion. These methods may be referred to as “image-domain” because they all require the computation of an imaging condition to generate an “image” (and often need to generate image gathers). The interferometric identity discussed in this disclosure above operates directly on extrapolated fields prior to any migration-type imaging step. Given that the evaluation of an imaging condition, in particular of extended image gathers (e.g., angle- or space/time-domain gathers), at many subsurface locations is very computationally demanding, our method does provide the advantage of yielding a subsurface-domain metric with no need for extended image gathers. Another difference is that any imaging condition requires a forward-modeled source wavefield in addition the extrapolated fields we discuss here.

Having established that the interferometric misfit differs from both data-domain FWI and image-domain tomography approaches, we find that there are many applications for the new metric.

An aspect of the misfit based on the interferometric identity is its implicit dependency on different in- and out-going waves from the data when they are extrapolated in a forward- or reverse-time fashion. This very particular aspect allows for different choices of medium configurations outside a bounded domain of interest. In fact, for data acquired in a medium that is heterogeneous both inside and outside of a target bounded region, it is shown that the extrapolation medium may be chosen outside the boundary to be completely homogeneous and the metric remains sensitive only to the parameters in the interior of the bounded domain. In practice, given boundary data acquired (or estimated, see below) in a fully heterogeneous subsurface, this would allow for waveform inversion of the medium properties in a target domain with no knowledge of the parameters outside of it. This target-oriented inversion capability, dependent only on the interior of a target bounded domain, is one beneficial aspect of the metric.

To use the metric, the target needs to be enclosed by a boundary in a geometry such as system 200 shown in FIG. 2 or system 400 in FIG. 4a. In practice, it may be difficult to have receivers at a bottom boundary (e.g., 252 in FIG. 2) to acquire data at the bottom boundary. This “missing boundary data” can be derived from the known boundary data (e.g., data at top boundary 240) using some methods, e.g., a redatuming scheme or Marchenko-type integral relation based method. In this case, the “missing boundary data” and the “model” are solved simultaneously based on an inversion process using the metric.

In addition to requiring enclosing boundaries, the extrapolation schemes require the acquisition of fields and their spatial derivatives on the boundary (e.g., pressure and its gradient, in the case of acoustic media): this is essential to the proper treatment of the in- and out-going wave constituents during extrapolation, particularly in heterogeneous media. This has two practical implications. First, the acquired data has all the necessary components; this is the case for, e.g., four-component (4C) ocean-bottom data (OBC). Secondly, all of the data components must be handled accordingly by the wavefield extrapolation schemes, both in forward- and reverse-time extrapolation. In the case in accordance with the present disclosure, this may be done by means of vector-acoustic wavefield extrapolation. Alternatively, one could also rely on the boundary data decomposed into in- and out-going components, which are then to be handled by appropriately modified extrapolators that can account for directional wavefield injection.

As an alternative to directly observed boundary data, estimated boundary data may be used. While it is possible to think of direct modeling of the boundary data given an a priori model, that would lead to a metric similar to J but with all data components dependent on the model; such a metric will have similar ill-posedness issues as those discussed above. A far more attractive proposition is to estimate the enclosing boundary data with no knowledge of the details of the subsurface. This is achievable by means of the data-driven, Marchenko-based iterative schemes. These methods allow for the iterative reconstruction of the true-amplitude, full-waveform response between a physical source and a virtual receiver inside the surface starting from one-sided surface data alone.

It is noted that the correlation- and convolution-type reciprocity integrals that are the basis for the interferometric identity in Eqs. 1 and 2 have known counterparts for elastic and electromagnetic waves in arbitrary inhomogeneous, anisotropic media. As such, as long as the geometric configuration used here remains the same, our metric extends to elastic and electromagnetic waves in arbitrary lossless media in a straightforward manner. In each case, however, one needs to select the proper combination of boundary data and extrapolator quantities so that extrapolation properly handles in- and out-going wave contributions. In addition, the interferometric identity may also, in principle, be extended to the case of attenuative media: this would involve the addition of the appropriate volume integrals to retrieve the extrapolated fields, and there would be an additional volume integral term in the J-metric in Eq. 7 that accounts for local attenuation properties. The extensions of our proposed misfit to these other types of media are also feasible.

### Inversion Based on the Metric

The metric as defined in Eq. 6 or similar quantifies the error between the model and the target object it represents. Therefore, the metric J may be treated as a cost function in a non-linear inversion of the target.

In accordance with one embodiment of the present invention, an inversion method 800 may include the following steps:

At step 810, collecting/receiving data;

At step 820, using the method 700 to form the cost function, which is the result from step 740, which is the identity of Eq. 6 or its variation; and

At step 830, minimizing the cost function with respect to the model using an optimization scheme.

Optionally, the inversion method may further include other steps:

At step 830, include any necessary preconditioning and/or regularization terms as in common practice in optimization; e.g., model smoothness constraints, bounds on model parameters, independent constraints (e.g., values from well-log data), data filters, etc. Alternatively, at step 830, minimizing the cost function (test value) with respect to the model using a multiscale optimization scheme, wherein the test value is a cost function for the optimization scheme for a given frequency or small subset of frequencies from which another model is derived at a given scale and then added to the model that is then used for a subsequent inversion using the next frequency sample or next set of frequencies.

At step 840, deriving additional metrics; and

At step 850, jointly optimizing all cost functions or metrics.

The above inversion methods are non-linear and the computation in the steps above can be intensive and costly. To reduce time and effort, some of the steps can be linearized. For example, the metric in Eq. 9 may be used for linearized migration-type imaging and inversion. In Eq. 9, part of the integrant δĜ_{A} (m), which is the perturbation of the extrapolator, is a known non-linear function of the model m or all its parameters. To approximate the perturbation of the extrapolator, a known nonlinear function of model parameters m, with a linear function with respect to a perturbation of model parameters m, whose derivative or integral are still linear functions of m, is a straightforward process. By doing this, the non-linear objective function J of Eq. 9 becomes a linear function of m. The non-linear inversion problem is simplified into a linear inversion problem. Besides the reduction in complexity and cost, the linear inversion problem is also more robust and less sensitive to the data noises.

This simplified and linearized method 900 may include steps of:

At step 910, linearizing the metric in a model perturbation,

At step 920, using the metric for a direct migration-type imaging using the associated sensitivity kernels.

In addition, and optionally, at step 930, the metric is used for linear inversion by, for example, least-squares linear imaging using the optimization objective of Eq. 8 for the linearized misfit.

The linearized method may optionally include a step 940, combining another metric or existing method for linear inversion, either in joint inversion or as inversion constraints.

As mentioned before, for simplicity, all examples discussed above are in the seismic imaging fields. The metrics discussed and the methods for using such metrics are applicable to any propagating wavefields. The waves may be acoustic (sound, ultrasound), elastic or electromagnetic waves (e.g., radio wave, microwave, X-ray). The measurement or acquisition geometry may be fully enclosing (e.g., bounded by boundaries 240 and 252), or partially enclosing (e.g., bounded by upper boundary 240 alone where bottom boundary 254 is at infinity). The acquisition system may be a seismic acquisition and imaging system, a medical acquisition and imaging system, a sonar acquisition and imaging system, a radar acquisition and imaging system, a satellite remote sensing acquisition and imaging system or a non-destructive engineering acquisition and imaging system, etc.

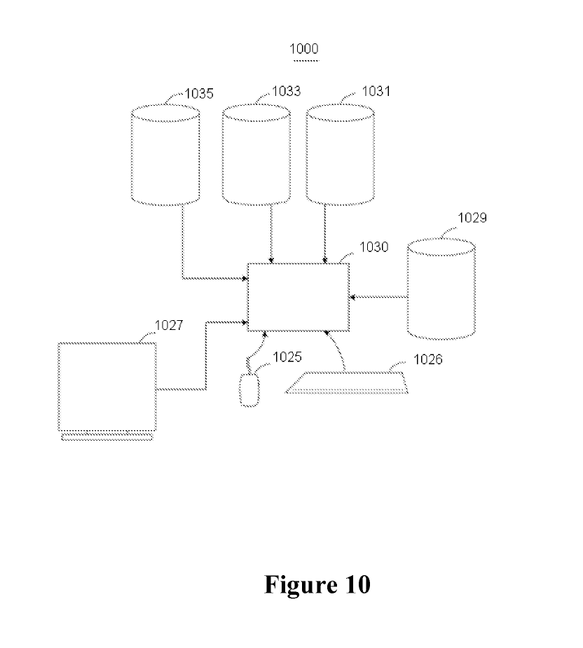

As those with skill in the art will understand, one or more of the steps of methods discussed above may be combined and/or the order of some operations may be changed. Further, some operations in methods may be combined with aspects of other example embodiments disclosed herein, and/or the order of some operations may be changed. The process of measurement, its interpretation and actions taken by operators may be done in an iterative fashion; this concept is applicable to the methods discussed herein. Finally, portions of methods may be performed by any suitable techniques, including on an automated or semi-automated basis on computing system 1000 in FIG. 10.

The methods described above are typically implemented in a computer system 1000, one of which is shown in FIG. 10. The system computer 1030 may be in communication with disk storage devices 1029, 1031, 1033 and 1035, which may be external hard disk storage devices. It is contemplated that disk storage devices 1029, 1031, 1033 and 1035 are conventional hard disk drives, and as such, will be implemented by way of a local area network or by remote access. Of course, while disk storage devices are illustrated as separate devices, a single disk storage device may be used to store any and all of the program instructions, measurement data and results as desired.

In one implementation, seismic data from the seismic receivers may be stored in disk storage device 1031. Various non-seismic data from different sources may be stored in disk storage device 1033. The system computer 1030 may retrieve the appropriate data from the disk storage devices 1031 or 1033 to process data according to program instructions that correspond to implementations of various techniques described herein. The program instructions may be written in a computer programming language, such as C++, Java and the like. The program instructions may be stored in a computer-readable medium, such as program disk storage device 1035. Such computer-readable media may include computer storage media. Computer storage media may include volatile and non-volatile, and removable and non-removable media implemented in any method or technology for storage of information, such as computer-readable instructions, data structures, program modules or other data. Computer storage media may further include RAM, ROM, erasable programmable read-only memory (EPROM), electrically erasable programmable read-only memory (EEPROM), flash memory or other solid state memory technology, CD-ROM, digital versatile disks (DVD), or other optical storage, magnetic cassettes, magnetic tape, magnetic disk storage or other magnetic storage devices, or any other medium which can be used to store the desired information and which can be accessed by the system computer 1030. Combinations of any of the above may also be included within the scope of computer readable media.

In one implementation, the system computer 1030 may present output primarily onto graphics display 1027, or alternatively via printer 1028 (not shown). The system computer 1030 may store the results of the methods described above on disk storage 1029, for later use and further analysis. The keyboard 1026 and the pointing device (e.g., a mouse, trackball, or the like) 1025 may be provided with the system computer 1030 to enable interactive operation.

The system computer 1030 may be located at a data center remote from an exploration field. The system computer 1030 may be in communication with equipment on site to receive data of various measurements. The system computer 1030 may also be located on site in a field to provide faster feedback and guidance for the field operation. Such data, after conventional formatting and other initial processing, may be stored by the system computer 1030 as digital data in the disk storage 1031 or 1033 for subsequent retrieval and processing in the manner described above. While FIG. 10 illustrates the disk storage, e.g. 1031 as directly connected to the system computer 1030, it is also contemplated that the disk storage device may be accessible through a local area network or by remote access. Furthermore, while disk storage devices 1029, 1031 are illustrated as separate devices for storing input seismic data and analysis results, the disk storage devices 1029, 1031 may be implemented within a single disk drive (either together with or separately from program disk storage device 1033), or in any other conventional manner as will be fully understood by one of skill in the art having reference to this specification.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope of the invention. Accordingly, the protection sought herein is as set forth in the claims below.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. §112, paragraph 6 for any limitations of any of the claims herein, except for those in which the claim expressly uses the words ‘means for’ together with an associated function.

1. A computer implemented method having a model of a target object, wherein the target is enclosed by a boundary and investigated by propagating a wave affected by the target, wherein the propagating wave is generated by at least one source outside the boundary, and the propagated wave is recorded by at least one receiver on the boundary, the method comprising:
obtaining/receiving wave measurement data at/from the boundary (710);
for a test location inside the target, by the computer,
deriving at least one reverse-time value by performing reverse time extrapolation of the boundary data to the test location using the model (720);
deriving at least one forward-time value by performing forward time extrapolation of the boundary data to the test location using the model (730); and
obtaining at least one test value by subtracting the forward-time value from the reverse-time value, wherein the test value indicates an inaccuracy of the model (740).

2. The method as in claim 1, further comprising:
obtaining test values for all locations within the target; wherein negligible magnitudes of all test values indicate the model is accurate at all locations within the target (750).

3. The method as in claim 2, further comprising:
evaluating the model for accuracy using the test values wherein the target is an object in:
a seismic acquisition and imaging system,
a medical acquisition and imaging system,
a sonar acquisition and imaging system,
a radar acquisition and imaging system,
a satellite remote sensing acquisition and imaging system, or
a non-destructive engineering acquisition and imaging system.

4. The method as in claim 1,
wherein the wave measurement data at the enclosing boundary are acquired by receivers on the enclosing boundary;
wherein the wave measurement data at the enclosing boundary are virtual data derived from measurement data acquired at locations other than the enclosing boundary; or
wherein the wave measurement data at the enclosing boundary comprise actual data acquired on the enclosing boundary and virtual data at the enclosing boundary derived from measurement data acquired at locations other than the enclosing boundary.

5. The method as in claim 1, wherein performing forward-time extrapolation or performing reverse-time extrapolation is done by:
explicitly computing impulse responses of an extrapolator;
implicitly computing impulse responses using wavefield injection; or
using exact reciprocity integral formulations,
wherein the computation is performed in time-domain or frequency domain.

6. The method as in claim 5, wherein the extrapolator is a full-wave-equation based extrapolator, a beam-based extrapolator or a ray-based extrapolator.

7. The method as in claim 1, wherein the model of the target is heterogeneous or homogeneous, isotropic or anisotropic, lossless or attenuative.

8. The method as in claim 1, further comprising:
minimizing the test value with respect to the model using an optimization scheme, wherein the test value is a cost function for the optimization scheme (830); or
minimizing the test value with respect to the model using a multiscale optimization scheme, wherein the test value is a cost function for the optimization scheme (830) for a given frequency or small subset of frequencies from which a second model is derived at a given scale and then added to the model that is then used for a subsequent inversion using the next frequency sample or next set of frequencies.

9. The method as in claim 8, further comprising:
deriving at least one metric (840); and
jointly optimizing the cost functions and the at least one metric (850).

10. The method as in claim 8, wherein the enclosing boundary comprises a top boundary and a bottom boundary.

11. The method as in claim 10, wherein the measurement data at the top boundary are acquired by receivers at the top boundary and the measurement data at the bottom boundary are missing;
wherein the missing data at the bottom boundary are estimated using the model; and
wherein minimizing the test value with respect to the model comprising jointly updating the model and estimating the missing boundary data.

12. The method as in claim 11, wherein the cost function has the form of Eq. 8 or Eq. 10.

13. The method as in claim 8, further comprising:
linearizing the metric in the model perturbation (910); and
using the metric for a direct migration type imaging using the associated sensitivity kernels (920).

14. The method as in claim 1, wherein the wavefield is an acoustic wavefield, an elastic wavefield or an electromagnetic wavefield.

15. A data processing system for processing imaging data, the system comprising:
at least one processor and at least one computer readable storage wherein the computer readable storage comprises computer executable instructions, which when executed by the processor, causes the processor to perform a method as in claim 1.
